(19) United States
(12) Patent Application Publication
(10) Pub. No.: US 2023/0290450 A1
(43) Pub. Date: Sep. 14, 2023

(54) ANIMAL PATIENT INSPECTION SUGGESTION SYSTEM, ANIMAL PATIENT INSPECTION SUGGESTION TERMINAL, AND ANIMAL PATIENT INSPECTION SUGGESTION PROGRAM

(71) Applicant: PECO INC., Tokyo (JP)

(72) Inventor: Jun OKAZAKI, Tokyo (JP)

(21) Appl. No.: 17/906,959

(22) PCT Filed: Mar. 23, 2020
(86) PCT No.: PCT/JP2020/012788

§ 371 Date: Mar. 31, 2023

(51) Int. Cl. G16H 10/60

(52) U.S. Cl. CPC G16H 10/60

(57) ABSTRACT

A simplified and highly accurate animal patient proposal system that solves a veterinary problem. An animal patient examination proposal system includes: a storage means that stores: an animal patient database including at least two or more pieces of medical interview information, physical examination information, present disease history information, examination history information, diagnosis history information, treatment history information, medication history information, and prescription history information, and an examination database including an examination candidate to be considered in association with each piece of information included in the animal patient database and an evaluation value associated with the examination candidate, an extraction means that refers to the examination database based on the patient database and extracts the examination candidate based on the evaluation value, and a display means that displays the extracted examination candidate to a user.

### TECHNICAL FIELD

The present invention relates to an animal patient examination proposal system, an animal patient examination proposal terminal and an animal patient examination proposal program which perform an examination proposal unique to the animal patient.

### BACKGROUND ART

Conventionally, various techniques have been proposed as a solution (so-called electronic medical record system) for electronically processing medical examination information of a human patient.

For example, Patent Literature 1 discloses a method for supporting a series of tasks including reception, preliminary examination, medical interview, medical service fee calculation, and accounting at medical institutions such as hospitals and clinics, nursing facilities, patients' homes and the like.

Meanwhile, such an electronic medical chart system may be time consuming for those unfamiliar with handling the system. Therefore, a technique for reducing the input work of such an electronic medical chart system has been proposed.

The technology described in Patent Literature 2 and Patent Literature 3 also relates to such an electronic medical record system, which is a technique for reducing the load of input work by predicting and displaying the content to be input in advance.

Further, Patent Literature 4 discloses a technique for extracting and estimating a patient group having a relatively high development risk to a specific disease among a plurality of patients, and Patent Literature 5 discloses a technique for estimating abnormality (diagnosis) from anamnesis and vital data. Both techniques aim to prevent input of the operator from making impossible in that some kind of estimation is displayed based on the inputted information.

### PRIOR ART LIST

### Patent Literature

Patent Literature 1: Japanese Unexamined Patent Publication No. 2017-191389
Patent Literature 2: Japanese Unexamined Patent Publication No. 2004-213678
Patent Literature 3: Japanese Unexamined Patent Publication No. 2017-162037
Patent Literature 4: Japanese Unexamined Patent Publication No. 2019-86839

### SUMMARY OF THE INVENTION

### Technical Problem

However, if the subject to be examined is an animal rather than a human, examinations, diagnoses, treatments, medications, and prescriptions that can be adapted depending on the animal species and individual differences may be fundamentally different, and provision of a new electronic medical record system reflecting these is expected.

Therefore, in the field of veterinary medicine as above, an object of the present invention is to provide an electronic medical chart system that is simplified, highly accurate, and also solves problems unique to the medical field.

### Technical Solution

According to the present invention, there is provided an animal patient examination proposal system comprising:
a storage means that stores an animal patient database including at least two or more pieces of medical interview information, physical examination information, present disease history information, examination history information, diagnosis history information, treatment history information, medication history information, or prescription history information, and an examination database including multiple examination candidates for animal patients;
an extraction means that refers to the examination database based on the patient database and extracts an examination candidate based on the evaluation value associated with each of the corresponding examination candidates; and
a display means that displays the extracted examination candidate to a user in a predetermined order.

### Advantageous Effects

According to the present invention, an animal patient proposal system that is more simplified, highly accurate and also solves a veterinary problem can be provided.

### BRIEF DESCRIPTION OF THE DRAWING

FIG. 1 is a block diagram showing the hardware configuration of a veterinary hospital terminal that executes an animal patient examination proposal system according to an embodiment of the present invention;

FIG. 2 is a functional block diagram of an animal patient examination proposal system according to an embodiment of the present invention;

FIG. 3 is a configuration example of an animal patient database;

FIG. 4 is an example of a data set (table) related to medical interview information contained in the examination database;

FIG. 5 is an example of a data set (table) related to physical information included in the examination database;

FIG. 6 is an example of a data set (table) related to current medical history information included in the examination database;

FIG. 7 is an example of a data set (table) related to diagnostic history information included in the examination database;

FIG. 8 is an example of a data set (table) related to epidemic diseases included in the examination database;

FIG. 9 is a processing flow diagram of the present system;

FIG. 10 is a conceptual diagram showing a method of updating the evaluation values of the examination database of the present invention;

FIG. 11 is a diagram showing a display example of the electronic medical chart system of the present invention;

FIG. 12 is a screen example of a state in which an examination candidate is recommended in the display of FIG. 11;

FIG. 13 is a screen example of a state in which the registration of the examination is accepted in the display of FIG. 11; and

FIG. 14 is a diagram showing a state in which the registered examination is displayed subsequent to FIG. 13.

### DETAILED DESCRIPTION OF THE EMBODIMENTS

Hereinafter, embodiments of the present invention will be described with reference to the accompanying drawings. Further, the embodiments described below do not unreasonably limit the content of the present invention described in the claims. Further, all components shown in the embodiments are not essential components of the present invention.

[Item 1]

An animal patient examination proposal system, comprising:
a storage means that stores: an animal patient database including at least two or more pieces of medical interview information, physical examination information, present disease history information, examination history information, diagnosis history information, treatment history information, medication history information, and prescription history information, and an examination database including an examination candidate to be considered in association with each piece of information included in the animal patient database and an evaluation value associated with the examination candidate;
an extraction means that refers to the examination database based on the patient database and extracts the examination candidate based on the evaluation value; and
a display means that displays the extracted examination candidate to a user.

[Item 2]

The animal patient examination proposal system according to Item 1,
wherein the examination database further includes information related to epidemic diseases or seasonal diseases, an examination candidate to be considered in association with each piece of information, and an evaluation value associated with the examination candidate.

[Item 3]

The animal patient examination proposal system according to Items 1 or 2, further comprising:
an evaluation value updating means that updates the evaluation value based on the selection operation by the user of the displayed examination candidate.

[Item 4]

The animal patient examination proposal system according to Item 3,
wherein the evaluation value updating means also updates the evaluation value based on the selection operation by another user on another terminal.

[Item 5]

The animal patient examination proposal system according to Items 3 or 4,
wherein the storage means stores attribute information of a user, and
the update degree for the evaluation value is determined based on the attribute information of the user.

[Item 6]

A portable animal patient examination proposal terminal, comprising:
a storage means that stores: an animal patient database including at least two or more pieces of medical interview information, physical examination information, present disease history information, examination history information, diagnosis history information, treatment history information, medication history information, and prescription history information, and an examination database including an examination candidate to be considered in association with each piece of information included in the animal patient database and an evaluation value associated with the examination candidate;
an extraction means that refers to the examination database based on the patient database and extracts the examination candidate based on the evaluation value; and
a display means that displays the extracted examination candidate to a user.

[Item 7]

An animal patient examination proposal program, making a portable animal patient examination proposal terminal having a touch display device function as:
a storage means that stores: an animal patient database including at least two or more pieces of medical interview information, physical examination information, present disease history information, examination history information, diagnosis history information, treatment history information, medication history information, and prescription history information, and an examination database including an examination candidate to be considered in association with each piece of information included in the animal patient database and an evaluation value associated with the examination candidate;
an extraction means that refers to the examination database based on the patient database and extracts the examination candidate based on the evaluation value; and
a display means that displays the extracted examination candidate to a user.

### SUMMARY

The animal patient examination proposal system (hereinafter referred to as “proposal system”) according to the embodiment of the present invention is particularly configured to accept operations by portable terminals that can be operated by veterinary workers such as veterinarians, other workers, or owners of animal patients, etc. (hereinafter collectively referred to as “veterinarians, etc.”) in animal hospitals.

The proposal system is mainly intended to enable the three steps of examination, diagnosis, and countermeasure (treatment, medication, and prescription) performed at animal hospitals to be processed on the same system, and in the present embodiment, a function of recommending a particularly appropriate examination will be described.

In particular, the proposal system is particularly configured to make use of an animal patient database that stores animal patient information, which is information related to an animal patient including information acquired before and after visiting the hospital, and an examination database that stores examination information that can be received at the veterinary hospital. The main purpose of the proposal system according to the present invention is to refer to animal patient information and examination information, extract at least one examination candidate that is most suitable for the animal patient, and provide it to the user.

### <Configuration>

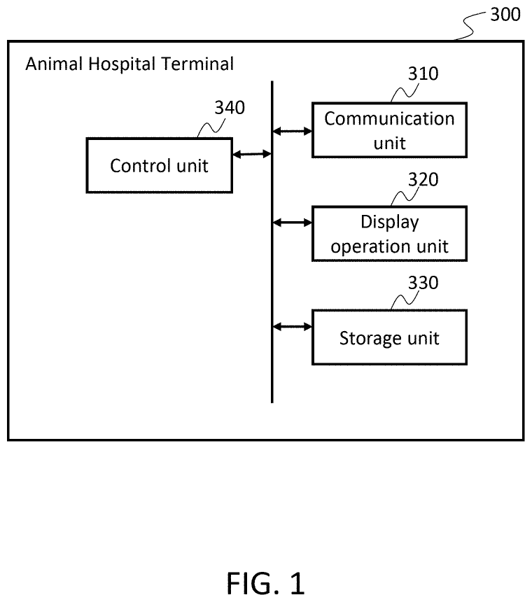

FIG. 1 is a block diagram showing the hardware configuration of a veterinary hospital terminal 300 that executes an animal patient examination proposal system according to an embodiment of the present invention. This proposal system is displayed via a display or the like of one or more animal hospital terminals

The animal hospital terminal 300 is an information processing device, such as a personal computer or a tablet terminal, which is managed by the animal hospital and used by the user described above, but may be configured with a smart phone, a mobile phone, a PDA, or the like. In particular, considering the fact that it can be easily carried in the hospital and the environment where the hair of animal patients comes into contact, a so-called tablet terminal is preferable.

Further, from a hygienic point of view, it is even more preferable to enable input operations using the voice recognition function. In this case, it is preferable that the voice recognition function is specialized for recognizing technical terms and abbreviations in the veterinary field.

Further, the proposal system according to the present embodiment describes that the function is completed only with the animal hospital terminal 300, but some functions can be performed on the server device or on the terminal of the animal patient's owner. In this case, part of the program executed in the animal hospital terminal 300 may be installed in the server terminal or the owner's terminal to logically configure and execute the entire proposal system.

### <Hardware Configuration of Animal Hospital Terminal 300>

As shown in FIG. 1, the animal hospital terminal 300 includes a communication unit 310, a display operation unit 320, a storage unit 330, and a control unit 340. In addition, the said structure is an example and may include another structure.

The communication unit 310 is a communication interface for performing communication with other terminals via a network, and can employ various communication protocols.

The display operation unit 320 is a user interface used to input an instruction by a user and display text, images, etc. according to input data from the control unit 340, and when the animal hospital terminal 300 is a tablet terminal, it is composed of a touch panel or the like. The display operation unit 320 is activated by a control program stored in the storage unit 330 and executed by the animal hospital terminal 300, which is a computer (electronic computer).

The storage unit 330 stores programs, input data, and the like for executing various control processes and functions in the control unit 340, and is composed of RAM, ROM, and other storage. Further, the storage unit 330 also temporarily stores communication content with other terminals.

The control unit 340 controls the overall operation of the veterinary hospital terminal 300 by executing programs stored in the storage unit 330, and is composed of a CPU, a GPU, and the like.

### <Functional Block of Examination Recommendation Module>

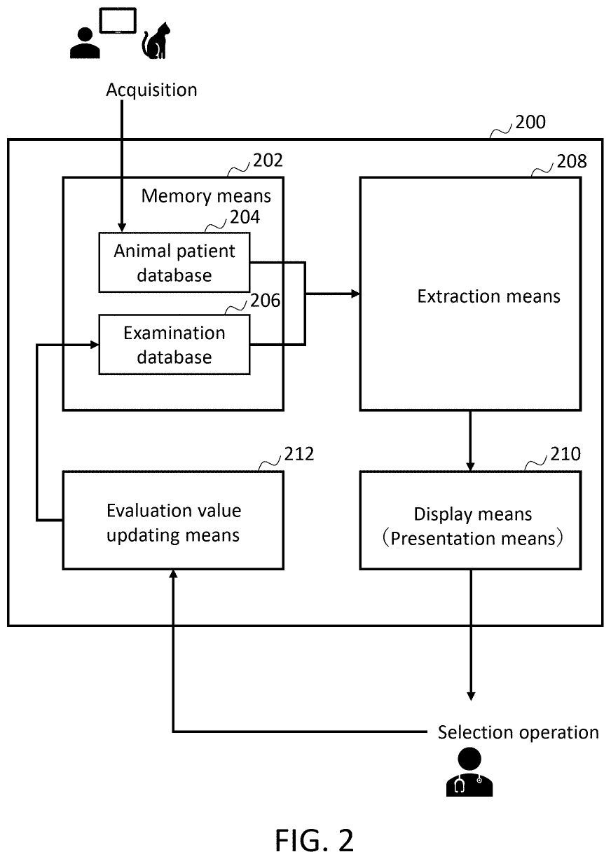

FIG. 2 is a functional block diagram showing the examination recommendation module 200 in the proposal system according to the present embodiment. The examination recommendation module 200 is composed of a function of recommending the optimum examination to be received for the animal patient based on the predetermined information input or obtained, and specifically, is a module in which the storage means, extraction means, and display means in FIG. 2 function together.

As shown, the examination recommendation module 200 has a storage means 202 containing an animal patient database 204 and an examination database 206. The storage unit 202 may store other information that configures the proposal system.

The animal patient database 204 stores current and past animal patient information managed by the proposed system according to the present embodiment. The animal patient information stored in the animal patient database 204 may include information on animal patients not only from the animal hospital concerned but also from other animal hospitals that are group-managed or group-controlled.

Meanwhile, the examination database 206 contains an examination candidate that can be performed on animal patients and their associated evaluation values.

The extracting means 208 refers to the examination database 206 based on the patient database 204 and extracts an examination candidate based on the evaluation values associated with the corresponding examination candidate. The extracted examination candidate is displayed to the user via display means such as a touch panel, for example, and receive a selection operation.

The display means 210 is a part of the display operation unit 320 in FIG. 1, and adopts a touch panel in the present embodiment. The display means 210 displays the extracted examination candidate to the user. In addition, since the system according to the present embodiment may be used in various places such as an operating room and an examination room in a veterinary hospital, a method of providing recommendation information and other notifications to the user may be an auditory provision method such as sound or warning sound rather than visual display. In this case, presentation of the voice or the like can be performed simultaneously with or separately from visual presentation.

The evaluation value updating means 212 also updates the evaluation value based on selection operations by other users on other terminals.

### <Details of Animal Patient Database>

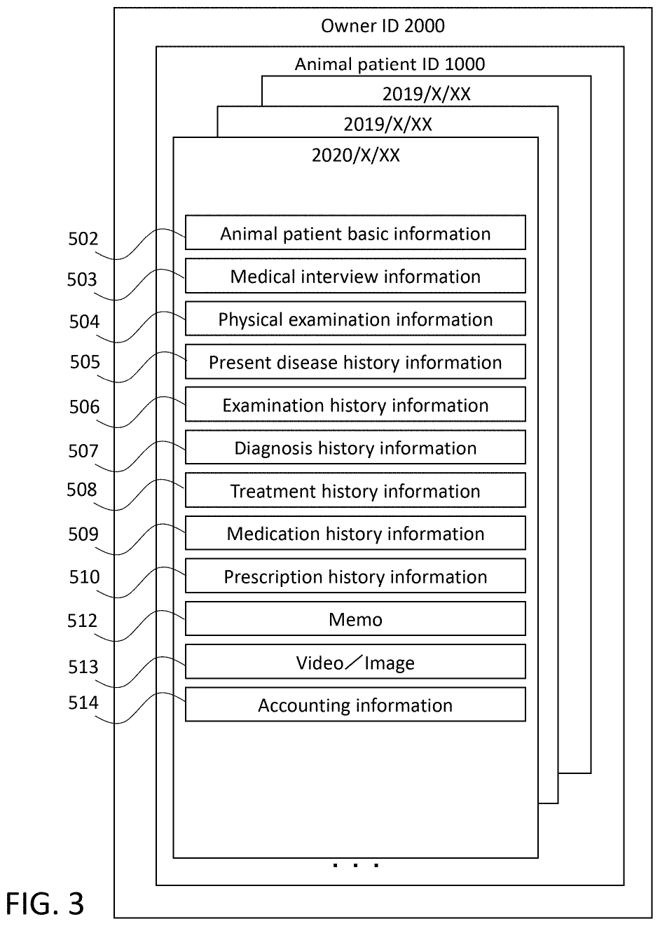

Each animal patient information included in the animal patient database 204 includes, for example, information as shown in FIG. 3. The animal patient database 204 of the present embodiment manages animal patients in association with animal patient IDs and owner IDs. Here, a case may occur in which a plurality of animal patient IDs could be associated with the same owner ID.

Medical chart data for each animal patient is managed for each medical examination reception day. For example, the electronic medical chart data of the user identified by the animal patient ID 1000 includes an animal patient basic information 502 such as name, gender, classification, size, medical interview information 503, physical examination information 504, present disease history information 505, examination history information 506, diagnosis history information 507, treatment history information 508, medication history information 509, prescription history information 510, memo 512, and information 513 such as video/image of an animal patient. Here, the input contents of the animal patient basic information 502, the inquiry information 503, and the information 513 such as moving images/images of the pet may be composed of data received from the portable terminal. Meanwhile, other animal patient information such as physical examination information can be composed of data received from the animal hospital terminal 300. Alternatively, it may be composed of data received by allowing a person in charge such as a veterinarian to input the information to the veterinary hospital terminal 300 based on information acquired from the owner of the animal patient.

In particular, the above-described animal patient basic information 502 includes animal species information to which the animal patient corresponds. Examples of animal species information include dog, fox, raccoon dog, fennec, cat, hamster, guinea pig, house mouse, chipmunk, Taiwan squirrel, prairie dog, flying squirrel, rabbit, monkey, miniature pig, ferret, skunk, donkey, horse (miniature horse, Falabella), hill myna, society finch, bengalee, budgerigar, cockatiel, Indian rose-necked parakeet, Java sparrow, pigeon, water turtle, tortoise, gecko, lizard, snake, crocodile, frog, salamander, goldfish, fancy carp, tropical fish, shrimp, beetle, stag beetle, bell cricket, aquatic insect, praying mantis, spider, scorpion, Thelyphonida, worm, sun spider, centipede, millipede and the like, but are not limited thereto. Furthermore, dogs may also have subordinate concepts such as dog breeds (breeds of dogs).

The medical interview information 503 is subjective information (chief complaint) acquired from the owner. For example, “I don't eat much”, “I have a scratch on my leg”, “I've gained weight”, “I'm feel down”, “I have a runny nose”, and the like, and is a symptom the owner observed when looking at an animal that the owner owns. The medical interview information 503 may be acquired from the owner using a medical interview form, a medical interview terminal, or the like when the animal visits the veterinary hospital. The medical interview information 503 may be obtained by having the owner enter it in advance using a portable terminal or the like owned by the owner.

The physical examination information 504 is physical information that can be examined by a veterinarian or the like at an animal hospital or the like and can be represented by objective numerical values. For example, it is “weight”, “body temperature”, “heartbeat”, “respiratory rate”, etc., and is information necessary for extracting necessary examinations together with present disease history information 505 described below.

The present disease history information 505 is subjective information acquired by a veterinarian or the like. The present disease history information 505 is often the same as the medical interview information 503 described above, and also includes, for example, the items that are difficult to actually check only with the owner saying “the neck is red”. Moreover, even if the information “blood in the urine” is acquired in the medical interview information 503, “bleeding from the anus” could be the current medical history when examined by a veterinarian or the like.

The examination history information 506 includes at least the history of examinations that the animal patient has undergone in the past. The examination history can include examinations that the patient has received in past outpatient visits, examinations that have been received in medical examinations, and the like. At that time, the reaction to the examination (information on the acceptability and admissibility of the examination itself, such as whether or not the person disliked it) may be associated and stored. This is because the information can be an important index when recommending an examination. The examination candidate may be categorized by purposes such as inflammation examination, biochemical examination, cardiac function examination, endocrine function examination, allergy examination, infectious disease examination, microbiological examination, morphological examination, and the like. The examination candidate may be categorized according to examination characteristics such as blood examination, X-ray examination, ultrasonography, cytological examination, endoscopy (gastric camera, rectal mirror), CT examination (imaging examination), pathological examination, and the like.

The diagnosis history information 507 includes at least the history of past diagnoses received by the animal patient. It can include information such as the names of specific diseases and injuries suffered in the past. The diagnosis history may include information regarding a diagnose suspected in a past visit.

The treatment history information 508 and the medication history information 509 include treatment history/medication history information that the animal patient received in the past. In this case, it is also possible to be stored in association with responses to the correspondence (information on the acceptability and tolerability of the treatment or medication itself, such as whether the patient disliked it, whether it affected their physical condition, etc.). Items include anal gland treatment, nail cutting treatment, sole treatment, ear treatment, hair removal, eye care, dental care, Elizabethan collars, abdominal bandages, etc., acupuncture and moxibustion treatment, indwelling, stool removal, suture removal, disinfection, oxygen treatment, vomiting treatment, emergency resuscitation measures, drainage, forced feeding, washing, pressure drainage, massage, microchip, trimming, catheterization, rehabilitation, surgery, anesthesia, hospitalization, injection, blood transfusion, IV infusion, examination, radiotherapy, various docks, nursing care, sanitary treatment, shampoo, training, supplements, snacks, contraception, castration, flea and tick extermination, rabies medicine, vaccines, foods, internal medicines, ear drops, nasal drops, eye drops, external medicine (ointment, cream), external medicine (disinfectant/spray), external medicine, filariasis prevention, and the like, but are not limited thereto.

The prescription history information 510 includes information such as drugs that have been prescribed to the animal patient in the past. In this case, information on the acceptability and tolerability of the treatment or medication itself, such as whether or not the patient was able to take the prescribed medication at home, whether the patient did not like it, whether it affected the patient's physical condition, etc. can be acquired via the user's portable terminal.

### <Details of Examination Database>

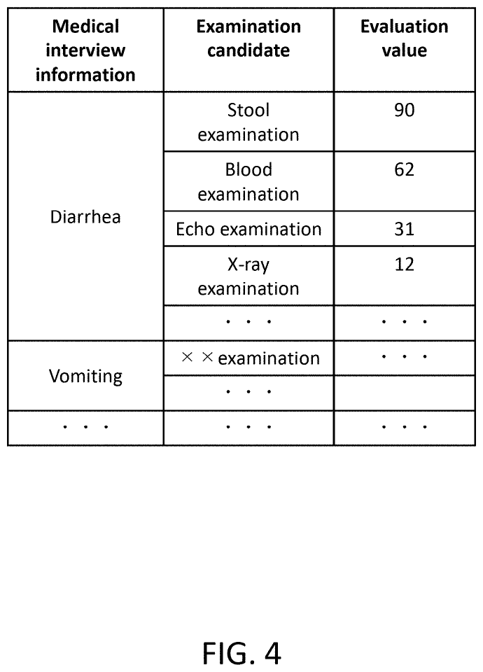

As shown in FIG. 4, the examination information database 206 has a data set (table) as illustrated in FIG. 4. The data set includes pairs of one or more examination names (examination candidates) associated with the information (medical interview information, present disease history information, physical examination information, etc.) included in the above-described animal patient information.

As illustrated, in the data set of the medical interview information in FIG. 4, one or more examination candidates (stool examination, blood examination, etc.) to be considered for the medical interview information (diarrhea, vomiting, etc.) and an evaluation value as the probability of performing each examination are maintained in association with each other.

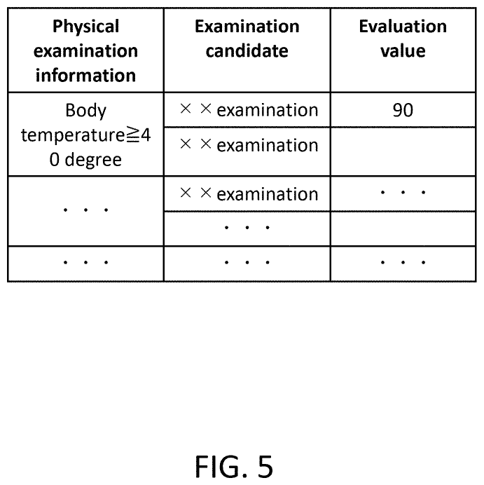

Further, in the physical examination information data set in FIG. 5, one or more examination candidates (X-ray examination, . . . etc.) to be considered for the physical examination information (body temperature ≥40 degrees, . . . etc.) and evaluation values as the likelihood of performing each examination are maintained in association with each other.

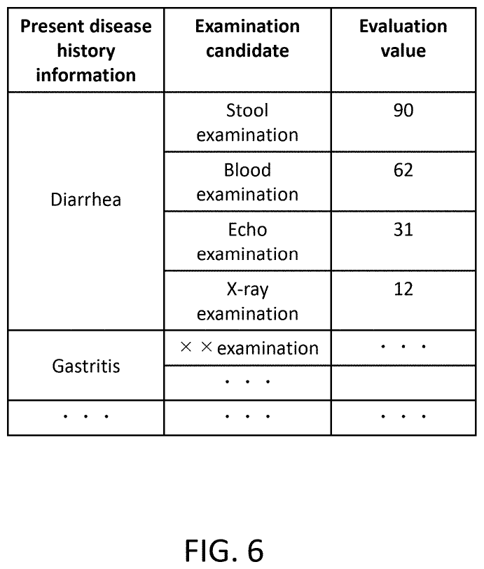

In the present disease history information data set in FIG. 6, one or more examination candidates (stool examination, blood examination, . . . etc.) to be considered for present disease history information (diarrhea, vomiting, . . . etc.) and an evaluation value as the likelihood of performing each examination are maintained in association with each other.

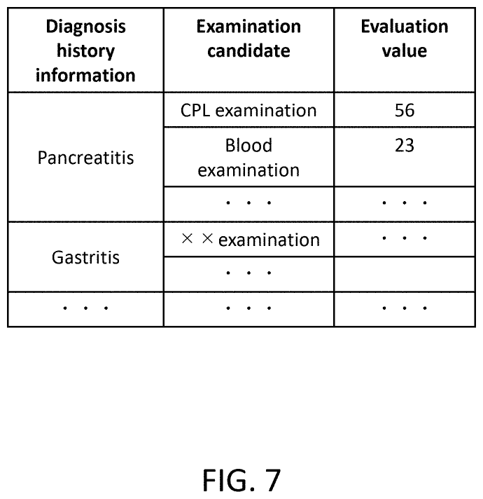

In the diagnosis history information data set in FIG. 7, one or more examination candidates (CPL examination, blood examination, etc.) to be examined for diagnosis history information (pancreatitis, gastritis, etc.) and an evaluation value as the likelihood of performing each examination are maintained in association with each other.

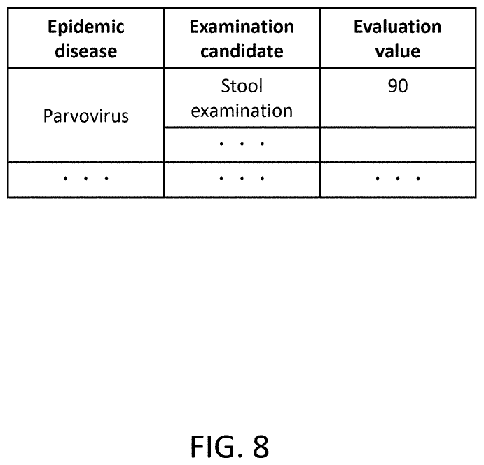

In the epidemic disease data set of FIG. 8, one or more examination candidates (stool examination, etc.) to be considered for epidemic diseases (parvovirus, etc.) and/or seasonal diseases and an evaluation value as the likelihood of performing each examination are maintained in association with each other.

The illustrated data sets are related to medical interview information, physical examination information, present disease history information, diagnosis history information, epidemic disease information or seasonal disease information, but information related to examination history information, treatment history information, medication history information, and prescription history information is also prepared. In this case, an examination candidate corresponding to each item and evaluation value as the likelihood of performing the examination are maintained in association with each other.

Further, the examination database 206 may include animal hospital equipment information or facility information related to the equipment, etc. as information unique to the animal hospital. The facility information is used to determine whether the examination can be performed at this hospital in the first place. As a result, for example, if there is no inspection equipment for performing a certain examination, the examination will not be recommended.

Further, the examination candidate may also be stored as a single examination candidate if multiple examination candidates are often combined, such as blood examination and X-ray examination.

### <Processing How>

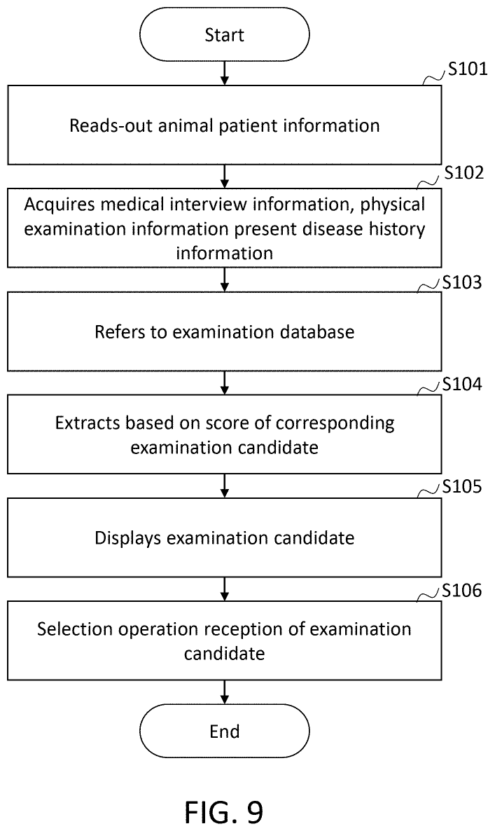

FIG. 9 is an example of a flowchart for explaining the flow of processing of the inspection recommendation module. The examination recommendation module reads out animal patient information from the animal patient database (S101), and acquires interview information, physical examination information, present disease history information, etc. (S102). After that, with reference to the examination database (S103), the corresponding examination candidates are extracted (S104).

Referring again to FIG. 9, the processing required up to extraction of the examination candidate is described in detail. For example, when the information “diarrhea” is acquired as medical interview information from a user who has visited the hospital, stool examination, blood examination, echo examination, and X-ray examination can be mentioned as examination candidates to be considered according to FIG. 4. After that, when a veterinarian, etc. examined the patient's present medical history and found it to be “diarrhea” in the same way, a stool examination, a blood examination, an echo examination, and an X-ray examination can be mentioned as examination candidates to be considered according to FIG. 6. In this case, the evaluation value of each of the stool examinations in the medical interview information and the present disease history information is 90, and when added together, it constitutes the highest evaluation value, so the stool examination is preferentially proposed as an examination candidate. Meanwhile, when referring to the diagnosis history information of the animal patient, if it is diagnosed as “pancreatitis” in the past, the evaluation values of the CPL examination and the blood examination are considered according to FIG. 7, and the priority of these examinations is increased as test candidates to be considered.

As the method of extracting examination candidates, it is also possible to simply add up the evaluation values, but information that should be considered more important when considering examination candidates may be weighted in a predetermined manner and then subjected to calculation such as summation. For example, in the above example, if the patient has been diagnosed with “pancreatitis” in the past, it is possible to raise the priority of proposals for examinations (such as the CPL examination in FIG. 7) necessary for determining whether pancreatitis has recurred. In such a case, as an extraction method, each evaluation value obtained by referring to each piece of information may be multiplied by a predetermined coefficient (weight). That is, the evaluation values obtained from FIGS. 4 to 8 (hereinafter referred to as evaluation values (a) to (e), respectively) may be multiplied by appropriate coefficients. For example, when each evaluation value is added up and an examination candidate corresponding to a higher evaluation value is preferentially proposed, it can be expressed as a mathematical formula in which α to δ are multiplied as arbitrary coefficients as follows.

Evaluation value (a)×α+Evaluation value (b)×β . . . Evaluation value (d)×δ

In this way, by proposing a plurality of examination candidates, it is possible to save labor in inputting examination names, etc., and to prevent omission of examination candidates from examination candidates to be considered. In particular, it is possible to correct and standardize the bias in judgment skills between those with many years of experience and those evaluated as having high achievements (referred to as “veteran veterinarians, etc.”) and those with few years of experience and those evaluated as not having high achievements (“novice veterinarians, etc.”).

The extracted examination candidate is displayed on the display (S105). At this time, the examination candidate with higher evaluation values may be displayed at higher ranks. The processing ends when the user selects one of the displayed examination candidates (S106).

In the present embodiment, the candidate extraction unit (temporary candidate extraction unit) may extract a plurality of examination candidates. In this case, for example, it is also possible to provide information on priority by displaying which examination is preferable together with a specific index (numerical value, rank, etc.).

### <Update Evaluation Value>

As shown in FIG. 2, the evaluation value updating means 212 may change/update the evaluation values included in the above-mentioned examination database 206 based on the user's selection operation of examination candidates. This can improve the accuracy of the recommendation function.

Further, the evaluation value updating means 212 may change/update the evaluation value based on selection operations by other users on other terminals (it does not matter whether it is another terminal in the same hospital or a terminal in another hospital connected to the same system).

The updating method of the evaluation value can be performed by various methods. As the simplest way of thinking, a method of increasing the evaluation value for the examination candidates that have actually been selected and conducted in the same case using the present system is considered. Meanwhile, the evaluation values of examination candidates that have been proposed but have not been selected or executed may be left unchanged or decreased.

The above-mentioned veteran veterinarians, etc. and novice veterinarians are stored in associated with a predetermined index (years of experience, etc.), and it is also possible to weight the increase/decrease of the evaluation value according to the selection operation by a veteran veterinarian or the like according to the index.

The evaluation value updating means 212 in the present embodiment not only updates the evaluation value itself, but also the weights (α to δ) given to the evaluation values when extracting the examination candidates described above and the index (years of experience) indicating the subject of the selection operation are also subject to update.

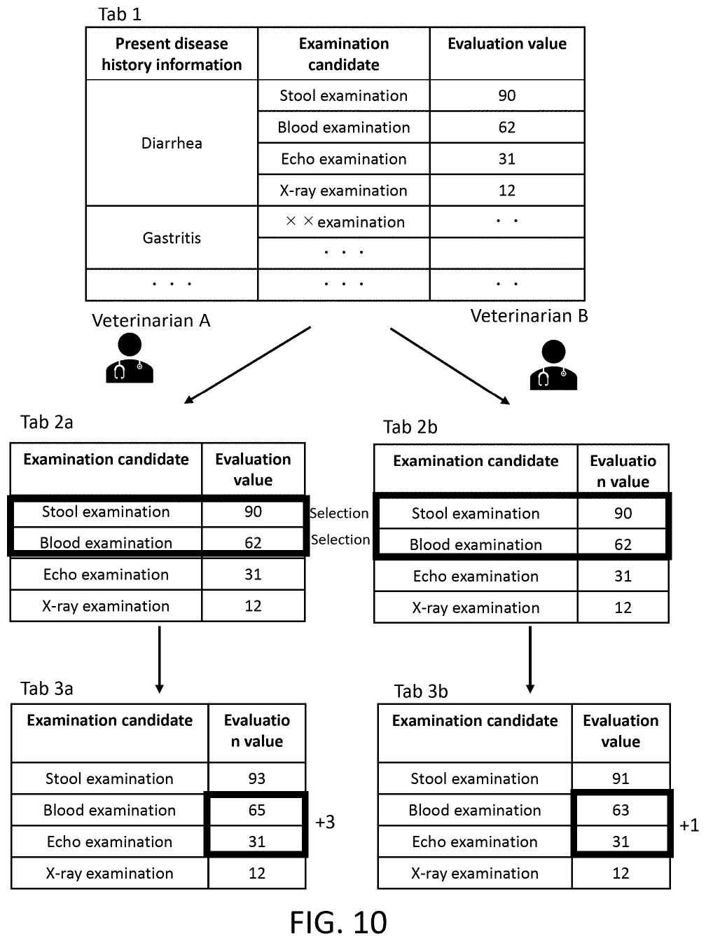

More specifically, as shown in FIG. 10, “stool examination, blood examination, echo examination, X-ray examination” corresponding to the present disease history information “diarrhea” in the current disease history information data set Tab1 of this system is specified as a recommendation candidate when, for example, “diarrhea” is identified during the interview of the present disease history at the time of medical examination.

At this time, in the terminals of veterinarian A and veterinarian B, who were each undergoing examination on different issues (of the same animal species), “stool examination, blood examination, echo examination, X-ray examination” were displayed as candidates (Tab2a, Tab2b). In the present embodiment, although the evaluation value itself is not displayed, an evaluation value or an alternative measure of certainty may also be displayed in some cases. As a result, it is possible to quantitatively convey the extent to which the examination is essential.

When the veterinarian A selects two of “stool examination, blood examination” from the displayed examination candidates, the corresponding examination order processing is started (not shown). Here, if veterinarian A is a veteran veterinarian, etc., and is registered in this system as having a high level of experience and achievements, from the idea that the “stool examination and blood examination” selected by veterinarian A may be given higher priority as an option in such cases, the evaluation values are updated so as to increase the evaluation values of these two examination candidates by +3 (Tab 3a).

Meanwhile, when the veterinarian B also selects two of the displayed examination candidates, i.e., “stool examination, blood examination”, the corresponding examination order processing is started (not shown). Here, in case where the veterinarian B is a novice veterinarian, etc., and is registered in this system as having little experience and track record, even if the “stool examination, blood examination” selected by the veterinarian B has a higher priority as an option in such a case, it is preferable that the amount of increase be smaller than that by a veteran veterinarian or the like. Therefore, the evaluation values are updated so as to increase the evaluation values of these two examination candidates by +1 (Tab 3b).

In the above explanation, the difference between veterinarian A and veterinarian B was whether they were veteran veterinarians or new veterinarians, but for example, the amount of update of evaluation values can be changed depending on whether the doctor is a specialist in the animal species to be examined. For example, when the subject to be examined is a so-called exotic animal (animal species other than dogs or cats), veterinarian A illustrated in FIG. 10 is a dog veterinarian and veterinarian B is an exotic animal specialist, it is preferable to raise the evaluation value of the examination candidate selected by veterinarian B.

Further, in the present embodiment, each data set associated with the animal species (see FIGS. 4 to 8, etc.) is prepared, and a corresponding data set is referenced based on the animal species included in the animal patient information. That is, for example, the content and range of the data set to be referenced when a diagnostic subject is a dog may be different from the content and range of the data set to be referenced when a diagnostic subject is an exotic animal.

### Display Example

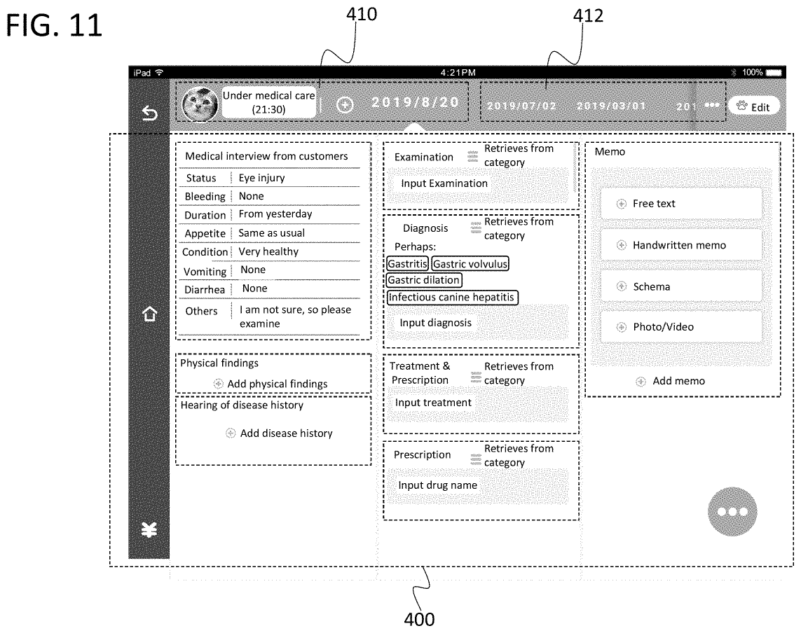

Subsequently, a display example of the proposal system will be explained. As shown in FIG. 11, for each animal patient of interest, the screen of the animal hospital terminal is composed of, at least, a medical data display region 400 that displays medical data generated for each medical interview reception date, and a date display range 410 that displays information indicating the medical interview reception date. Further, the region 410 may also display information such as an image of the animal patient, the clinical status of the animal patient (under medical care, under examination, completed medical care, completed examination), and the like.

In the medical data display area 400, medical data such as medical interview information (situation, bleeding, duration, appetite, physical condition, vomiting, diarrhea, etc.), physical findings, medical history, examination, diagnosis, treatment & medication, prescription, and memo are displayed so that they can be input. Further, as will be described later, an area in which information can be entered is provided for each item included in the medical care data, and the medical data is updated by input by a veterinarian or the like.

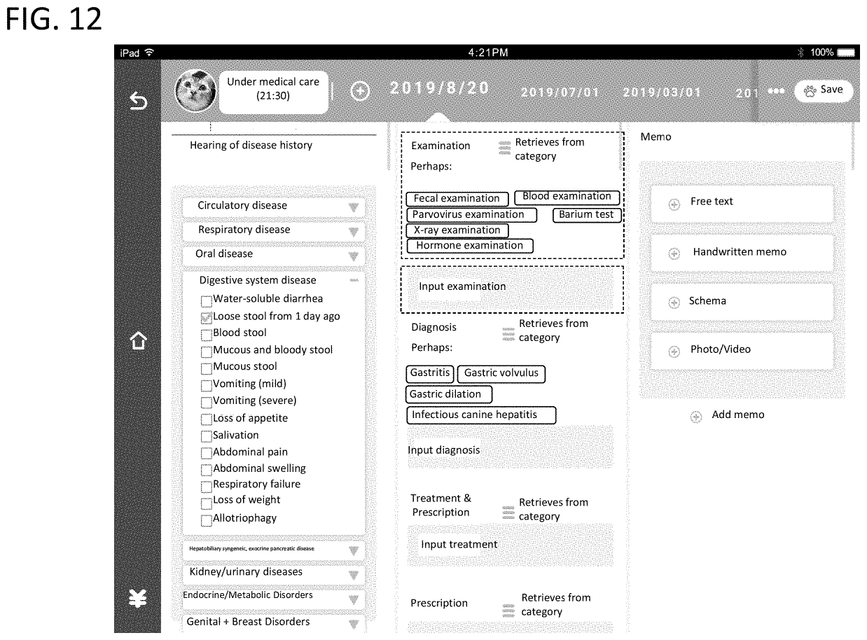

As shown in FIG. 12, when input of the medical interview, physical findings, and medical history is completed, examination candidates such as “stool examination, parvovirus examination, blood examination, X-ray examination, barium examination, hormone examination” are recommended and displayed in the examination area. As for the timing of the display, the recommendation function may be appropriately executed before the input of medical interview, physical findings, and medical history is completed.

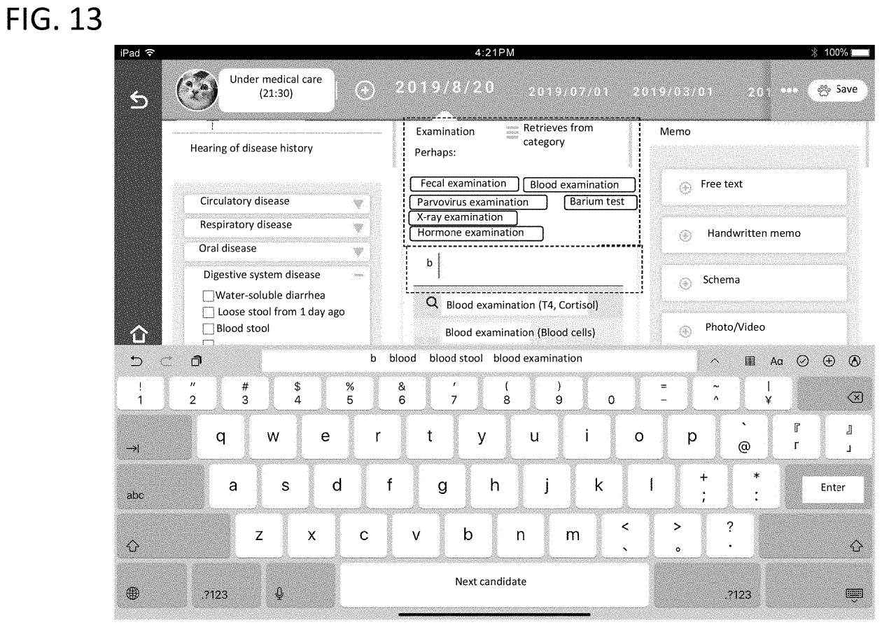

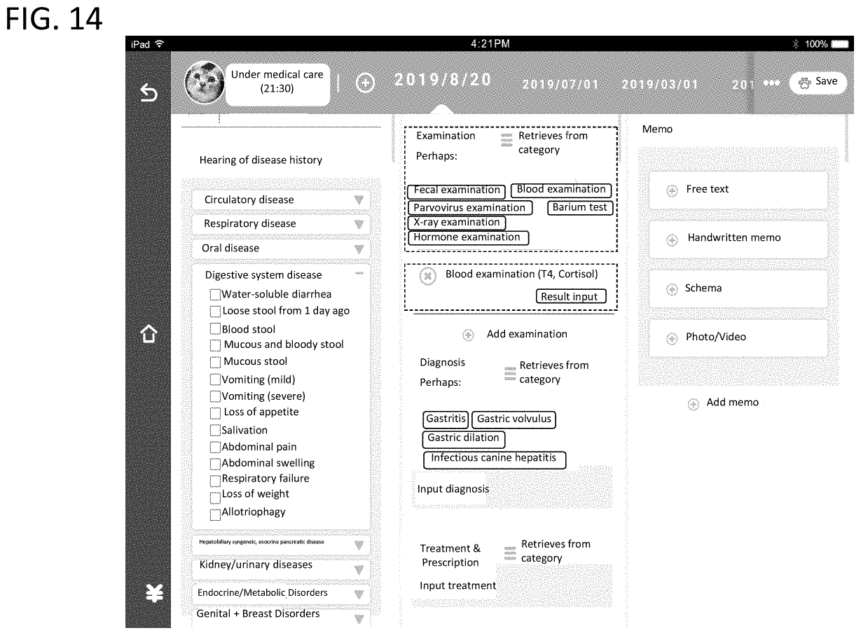

The examination can also be inputted as shown in FIG. 13. In this case, if “Ke” is entered, the examination can be predicted as “blood examination” (Ketsueki Kensa). When the examination is confirmed, the selected examination name is displayed in the examination area as shown in FIG. 14.

By providing the proposal system including the user interface as described above, a person in charge such as a doctor at a veterinary hospital can perform an integrated work together with the input of clinical data. Especially in the case of medical care with animals as patients, the tests, diagnoses, treatments, medications, and prescriptions that can be adapted to animal species and individual differences are fundamentally different, unlike human medical care, so that a proposal system that reflects them can be provided. Thereby, a proposal system that is more simplified and has higher accuracy, and can solve problems specific to the field of veterinary medicine can be provided.

Although embodiments according to the invention have been described above, these can be implemented in various other embodiments, and can be implemented with various omissions, replacements, and changes. These embodiments and variations as well as those with omissions, substitutions and modifications are included in the technical scope of the claims and the equivalent scope thereof.

### DESCRIPTION OF REFERENCE NUMERALS

200 Examination recommendation module
202 Storage means
204 Animal patient database
206 Examination database
208 Extraction means
210 Display means
212 Evaluation value updating means
300 Animal hospital terminal

1. An animal patient examination proposal system, comprising:
a storage means that stores: an animal patient database including at least two or more pieces of medical interview information, physical examination information, present disease history information, examination history information, diagnosis history information, treatment history information, medication history information, and prescription history information, and an examination database including an examination candidate to be considered in association with each piece of information included in the animal patient database and an evaluation value associated with the examination candidate;
an extraction means that refers to the examination database based on the animal patient database and extracts an examination candidate based on the evaluation value; and
a display means that displays the extracted examination candidate to a user.

2. The animal patient examination proposal system according to claim 1,
wherein the examination database further includes information related to epidemic diseases or seasonal diseases, an examination candidate to be considered in association with each piece of information, and an evaluation value associated with the examination candidate.

3. The animal patient examination proposal system according to claim 1, further comprising:
an evaluation value updating means that updates the evaluation value based on the selection operation by the user of the displayed examination candidate.

4. The animal patient examination proposal system according to claim 3,
wherein the evaluation value updating means also updates the evaluation value based on the selection operation by another user on another terminal.

5. The animal patient examination proposal system according to claim 3,
wherein the storage means stores attribute information of a user, and
the update degree for the evaluation value is determined based on the attribute information of the user.

6. A portable animal patient examination proposal terminal comprising:
a storage means that stores: an animal patient database including at least two or more pieces of medical interview information, physical examination information, present disease history information, examination history information, diagnosis history information, treatment history information, medication history information, and prescription history information, and an examination database including an examination candidate to be considered in association with each piece of information included in the animal patient database and an evaluation value associated with the examination candidate;
an extraction means that refers to the examination database based on the animal patient database and extracts the examination candidate based on the evaluation value; and
a display means that displays the extracted examination candidate to a user.

7. (canceled)

8. A method for proposing an examination for an animal patient executed by a computer, the method comprising:
storing an animal patient database including at least two or more pieces of medical interview information, physical examination information, present disease history information, examination history information, diagnosis history information, treatment history information, medication history information, and prescription history information, and an examination database including an examination candidate to be considered in association with each piece of information included in the animal patient database and an evaluation value associated with the examination candidate;
extracting the examination candidate based on the evaluation value referring to the examination database based on the animal patient database; and
displaying the extracted examination candidate to a user.
